(19) United States
(12) Patent Application Publication
(10) Pub. No.: US 2005/0148253 A1
(43) Pub. Date: Jul. 7, 2005

(54) Rapid absorbent structures

(75) Inventor: Evan E. Koslow, Weston, CT (US)

Correspondence Address: DELIO & PETERSON, 121 WHITNEY AVENUE, NEW HAVEN, CT, 06510, US

(73) Assignee: copy from parent application

(21) Appl. No.: 11/070,694

(22) Filed: Mar. 3, 2005

Related U.S. Application Data
(63) Continuation of application No. 09/864,976, filed on May 24, 2001.

(51) Int. Cl. B32B 27/00; B32B 27/14; B32B 27/12; A61F 13/15

(52) U.S. Cl. 442/118; 428/103; 428/118; 428/167; 428/171; 428/172; 428/402; 604/317; 604/358; 604/365

(57) ABSTRACT

A liquid absorbent composite is disclosed. The composite comprising a first substrate and a laminate or bonded mixture, the laminate or bonded mixture comprising a mixture of binder particles and super-absorbent polymer particles, wherein the binder particles are on average smaller than the super-absorbent polymer particles, wherein at least some of the binder particles coalesce at least some of the bonded mixture to the substrate, wherein at least some of the bonded mixture forms a three-dimensional array of elongated channels upon contact with a liquid.

### BACKGROUND OF THE INVENTION

1. Field of the Invention

The present invention relates to liquid absorbent composites, and more specifically to disposable liquid absorbent composites, including but not limited to, adult incontinence products, diapers, training pants and paper towel products aimed at absorbing liquids.

2. Description of the Prior Art

Manufacturers of disposable absorbent products often use thick, fluff pulp batting mixed with super adsorbent polymers (hereinafter SAP), to absorb and retain deposited liquids. Fluff pulp is a moderately effective absorbent, even when liquids are delivered in multiple challenges over short time intervals.

A disadvantage of using fluff pulp mixed with SAP is the overall thickness and bulkiness of the product. This bulkiness only increases once liquid has been absorbed by the article due to swelling of the SAP. This attribute is of particular concern for adult consumers using incontinence products.

In addition, fluff pulp pricing is highly volatile and thus represents a significant manufacturing risk. Fluff pulp price volatility has been responsible for substantial profit losses to absorbent product manufacturers.

Another disadvantage of using fluff pulp mixed with SAP as an absorbent is a phenomenon called “gel blocking” or “gelling”. SAP granules that are not properly distributed within the fluff pulp swell and coalesce upon exposure to liquid. In doing so, the swollen particles prohibit further absorption of liquid deposited in subsequent challenges.

Therefore, it would be desirable to utilize a highly absorbent, inexpensive and thin composite, without fluff pulp, in replacement of the thick, expensive, fluff pulp containing media presently used in disposable absorbent products.

The present invention provides for a thin, inexpensive, super absorbent composite for the rapid absorption of liquid by SAP particles, wherein containment of liquid occurs within a three-dimensional array of cavities, hollow tubes, folds, and elongated channels (hereinafter collectively referred to as “channels”) comprised of SAP particles, that are spontaneously formed when the SAP is contacted with liquid.

The present invention further provides for an absorbent composite with a dry thickness of less than approximately two millimeters, yet is highly absorbent and does not contain fluff pulp. Still further, the present invention provides a solution to the “gel blocking” phenomena prevalent in the prior art.

### SUMMARY OF THE INVENTION

The present invention is directed to an absorbent composite comprising a first substrate and a laminate or bonded mixture of binder particles and SAP particles, wherein the binder particles are on average smaller than the SAP particles, wherein at least some of the binder particles coalesce at least some of the bonded mixture to the substrate, wherein the bonded mixture spontaneously forms a three-dimensional array of elongated liquid receiving channels upon contact with a liquid.

Also disclosed is a method of absorbing liquid comprising the steps of:

a) placing a composite adjacent to a liquid source, wherein the composite comprises a first substrate and a laminate or bonded mixture, the bonded mixture comprising a mixture of binder particles and SAP particles, wherein the binder particles are on average smaller than the SAP particles, wherein at least some of the binder particles coalesce at least some of the bonded mixture to the substrate, and wherein the bonded mixture spontaneously forms a three-dimensional array of elongated channels upon contact with a liquid from a liquid source; and

b) absorbing the liquid with the bonded mixture.

In addition, the present invention describes a liquid absorbent pad, which comprises:

a) an outer layer of a substantially liquid-impervious material having an outer surface and an inner surface; at least one composite segment positioned on the inner surface of the liquid impervious material, the composite segment comprising a first substrate and a bonded mixture, the bonded mixture comprising a mixture of binder particles and SAP particles, wherein the binder particles are on average smaller than the SAP particles, wherein at least some of the binder particles coalesce at least some of the SAP particles to each other, and wherein at least some of the binder particles coalesce at least some of the SAP particles to the substrate, and wherein the SAP particles spontaneously form a three-dimensional array of elongated channels upon contact with a liquid; and

b) a liquid-permeable acquisition layer in liquid communication with the composite segment, wherein at least a portion of the outer layer and the liquid permeable acquisition layer are attached, and the composite segment is sandwiched therebetween.

This invention will be discussed in greater detail in the description that follows. Additional advantages of the invention will become apparent from this discussion, together with accompanying drawings.

### BRIEF DESCRIPTION OF THE DRAWINGS

FIG. 1 is a top view of the SAP composite of the present invention, with the top sheet removed, demonstrating the resultant three-dimensional array of channels spontaneously formed when the SAP composite of the present invention is put into contact with liquid.

FIG. 2 is a top view of a prior art SAP composite, with the top sheet removed, demonstrating the resultant planar structure that forms when a prior art SAP composite is put into contact with liquid.

FIG. 3 is a cross sectional view of the SAP composite of the present invention, with the top sheet removed, demonstrating the resultant three-dimensional array of channels spontaneously formed when the SAP composite of the present invention is put into contact with liquid.

FIG. 4 is a cross sectional view of a prior art SAP composite, with the top sheet removed, demonstrating the resultant planar structure that forms when a prior art SAP composite is put into contact with liquid.

FIG. 5 is a cross-sectional schematic of a preferred embodiment of the SAP composite of the present invention.

FIG. 6 is a schematic diagram illustrating an apparatus and process for making the SAP composite of the present invention.

FIG. 7 is a perspective view illustrating the apparatus used in testing the SAP composite of the present invention.

FIG. 8 is a graphical representation indicating measured performance results of the SAP composite of the present invention, compared to measured results of a prior art SAP composite.

FIG. 9 is a greatly enlarged perspective view of a portion of a liquid absorbent pad constructed in accordance with the present invention, partially separated to reveal the internal construction.

### DETAILED DESCRIPTION OF THE INVENTION

This invention is the result of the discovery of an unexpected and novel phenomena. It has been discovered that use of a SAP, that when processed into a laminate, has the capacity to spontaneously undergo a rapid change from a thin planar laminate, into a three-dimensional array of cavities, hollow tubes, folds, and elongated channels upon exposure of the laminate to liquid to produce superior liquid absorbent composites. A preferred process for forming the present invention is described in U.S. Pat. No. 6,015,608 to Koslow, the disclosure of which is incorporated herein by reference.

Formation of this three-dimensional array of channels in the SAP laminate that spontaneously form when the laminate is put in contact with liquid, cause liquid to be absorbed at exceptional speed, often within 1 to 4 seconds, as compared to the prior art SAP composite. The formation of these channels allow for rapid bulk flow of liquid into the channels, where the liquid is then absorbed by the SAP particles.

Although unknown, it is believed that the formation of channels in the SAP upon contact with liquid is the result of liquid induced asymmetric stresses that develop within the immobilized laminate. These stresses cause sections of the SAP laminate to spontaneously produce channels of a generally well-defined size, generally approximately 0.15-0.30 inches in diameter.

As the spontaneously formed channels become flooded with additional incoming liquid, these channels envelope the liquid thus accomplishing an extremely rapid containment thereof. Generally within about an additional 15-25 seconds, the contained liquid is fully absorbed within the SAP particles.

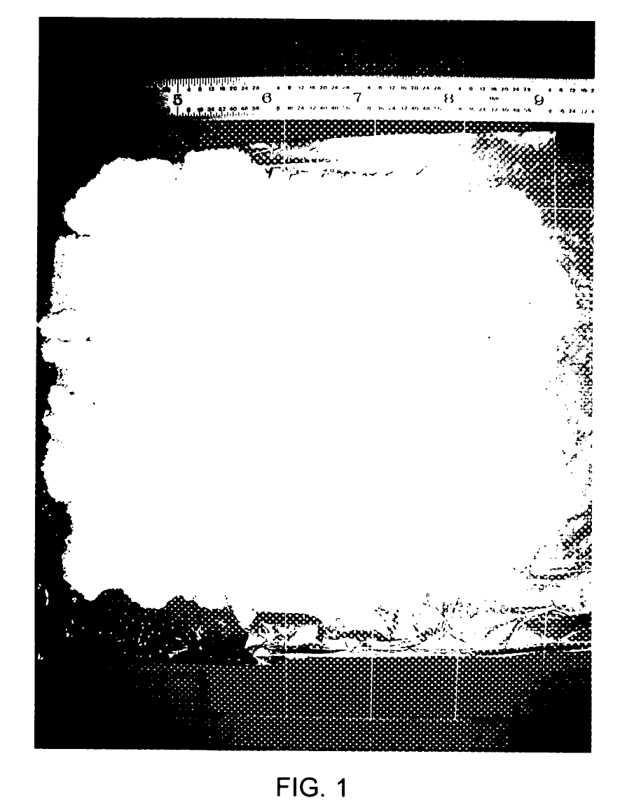

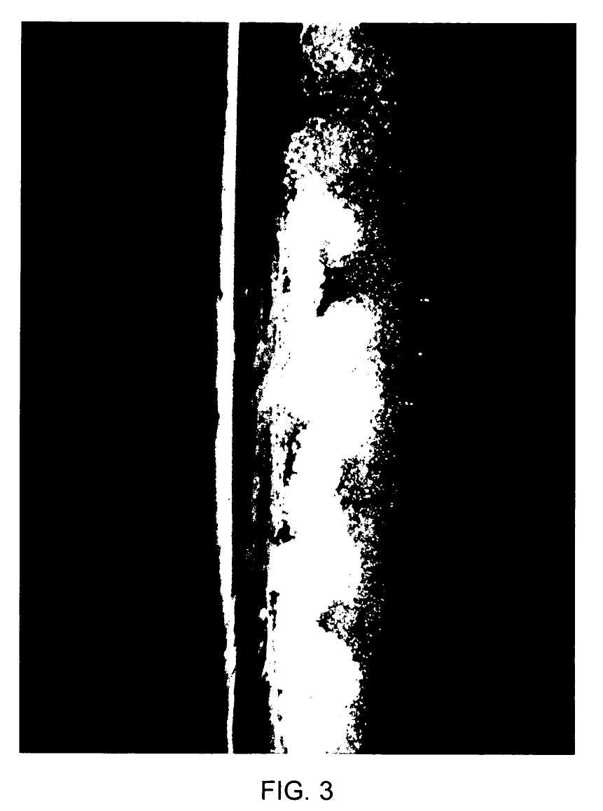

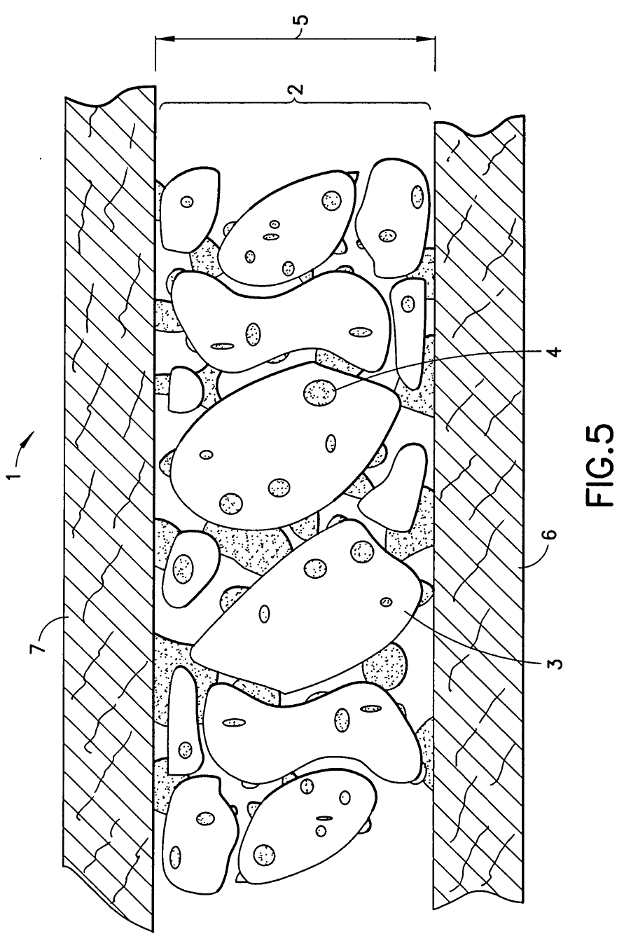

The spontaneous conversion of the immobilized bonded mixture of the relatively thin, planar laminate of SAP particles is demonstrated by viewing the laminate schematically shown in FIG. 5, compared to the array of three-dimensional channels shown in FIG. 1 and FIG. 3.

This phenomena is unique and unexpected. SAP particles, which do not possess this property when processed in accordance with the same process as is the SAP composite of the present invention, do not exhibit this enhanced liquid absorption property when brought into contact with liquid. One particular grade of SAP in which this property is observed, is SAP grade SP-1224 available from Stockhausen Corporation (Greensboro, N.C.).

Referring to the drawings and, in particular, FIG. 5, there is provided an absorbent composite generally indicated as 1. The composite 1 has a first substrate or backing layer 6 and optionally a second substrate sometimes referred to as a covering layer or top sheet 7. First substrate 6 and second substrate 7 may be formed of various materials depending upon the intended application and need not be formed of the same or similar material within one composite. By way of example only, substrates 6 and/or 7 may be permeable materials, such as non-woven fibrous webs, e.g., spun bonded, melt blown or carded materials composed of polyester or polyolefinic fibers. The substrates may also be formed from woven materials. Substrates 6 and/or 7 may optionally be formed wholly or in part from cellulosic materials including tissue or towel stock. In the alterative, substrates 6 and/or 7 may be either semi-permeable or impermeable to liquids, e.g., a polymeric film, although such impermeable substrates 6 and 7 would not typically be used at the same time. Usually, the first or second substrate of the laminate must be at least semi-permeable.

Coalesced with first substrate 6, and optionally with second substrate 7, is an absorbent laminate indicated generally as 2 in FIG. 5. Laminate 2 is comprised of SAP particles 3 having the channel forming properties of the present invention, and binder particles 4. The binder particles 4 coalesce at least some of the SAP particles 3. An amount of binder particles 4 also coalesce at least some of the SAP particles 3 to substrate 6, and optionally to substrate 7, or to both substrates 6 and 7.

Any suitable binder material may be employed in this invention. Materials suitable for forming binder particles 4 include, but are not limited to, thermoplastic or thermosetting binders. Preferred binder materials include, polyethylene, polypropylene, poly (ethylene vinyl acetate), and nylon.

The dry thickness of the composite 5 is defined as the thickness of the bonded mixture prior to the composite being put into contact with liquid.

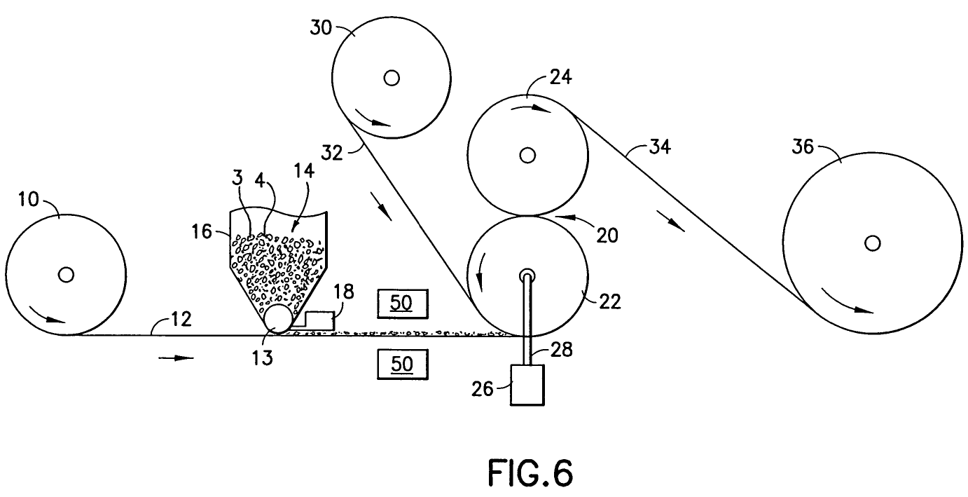

FIG. 6 illustrates an exemplary apparatus used to produce this invention. A supply roll 10 provides a first substrate 12. Downstream from supply roll 10 is a knurled roller 13 positioned to receive a mixture of SAP particles 3 and binder particles 4, generally indicated as mixture 14, from hopper 16. Mixture 14 is applied to the upper surface of substrate 12 as a continuous coating or, alternatively, as a coating in a specific design including, but not limited to, stripes. A brush 18 may be employed to aid in removing mixture 14 from knurled roller 13.

Thereafter, substrate 12 containing mixture 14 is passed through nip 20 between a heated idler roller 22 and a drive roller 24. Alternatively, before being passed through nip 20, substrate 12 containing mixture 14, may be preheated by a pre-heater 50 such as, for example a convection or infrared oven. A pneumatic cylinder 26 is connected via a rod 28 to the axle of idler roller 22 to maintain a desired pressure on substrate 12 containing mixture 14 within nip 20. In passing through pre-heater 50, and over the surface of heated roller 22, mixture 14 is heated to a temperature equal to or greater than the softening temperature of binder particles 4, but to a temperature below the softening temperature of SAP particles 3. Within nip 20, binder particles 4 coalesce under pressure with SAP particles 3. An amount of binder particles 4 coalesce with first substrate 12.

Furthermore, in a preferred embodiment of the present invention, a second supply roll 30 of a second substrate 32, which may be of the same or may be of a different material from that of substrate 12, is also passed between nip 20 on the top of mixture 14. An amount of binder particles 4 coalesce with second substrate 32. Upon leaving nip 20, binder particles 4 cool and harden. The finished composite 34 then passes onto take-up roll 36.

By selection of substrate materials 10 and 20, binder materials 4, SAP materials 3, SAP to binder weight ratios, absolute amounts of mixture 14 applied to substrate 12 per unit area, binder particle size, SAP particle size, the ratio of binder particle size to SAP particle size, heating temperature, nip pressure and linear speed of substrate, it is possible to vary composite depth, porosity, permeability, tensile strength, flexibility, pleatability, draping ability, wicking, internal liquid distribution, or other attributes of the SAP composite of the present invention.

The effective diameter range of binder particles is from about 5 to about 100 microns, preferable from about 5 to about 50 microns. The effective diameter of SAP particles is from about 5 to about 5000 microns.

Although unknown, it is believed that gel blocking in the SAP composite of the present invention is at least partially prevented by the presence of binder particles 4 between particles of SAP 3 having the channel forming properties of the present invention. This is especially true when binder particles 4 are slightly hydrophobic, present in a minority amount by weight, and are much smaller in size than the SAP particles 3. The binder particles 4, are preferably 4 to 25 times smaller in average diameter, than the SAP particles 3.

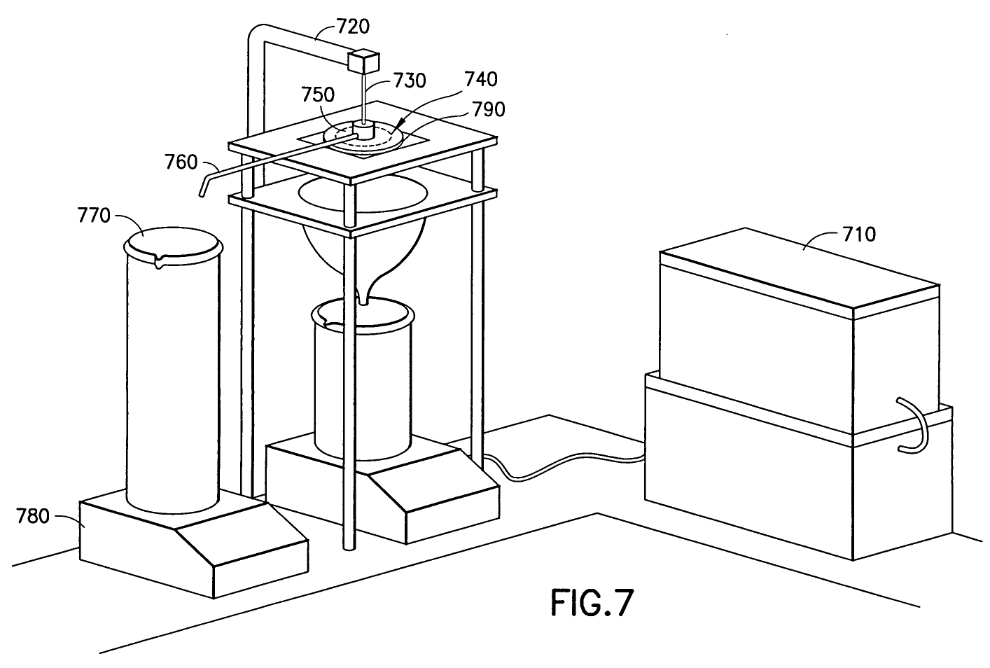

FIG. 7 illustrates an exemplary apparatus for testing the liquid absorption properties of the SAP composite of the present invention. A liquid contained within vessel 710, typically de-ionized water containing 1% by weight sodium chloride (similar to urine), is delivered to the sample medium 790, via pump system 720 and delivery tube 730. A liquid impermeable layer of plastic film is placed over hole 740. Sample medium 790 is placed directly over the plastic film. Two layers of a liquid permeable acquisition material are then placed directly over sample medium 790. The liquid permeable acquisition layers are generally composed of a carded, nonwoven web. A plate 750 with a 1.0″ diameter hollow chamber is then placed directly over the previously mentioned layers. Delivery tube 730 is inserted into the top receptacle of plate 750. This allows liquid delivered from delivery tube 730 to be released into the 1″ hollow chamber (sometimes known as the “drop zone”). A 4000 ml beaker 770 is placed on a balance 780 and tarred. Plate 750 is positioned as such that the outlet of overflow nozzle 760 is in alignment with the top of beaker 770. When liquid from vessel 710 is released into the hollow chamber of plate 750, the liquid is either rapidly absorbed by the sample medium 790, or it accumulates within the hollow chamber of plate 750. Liquid that accumulates and is not absorbed by the sample, overflows through nozzle 760 and is collected in beaker 770, the mass of which is recorded on balance 780 as a function of time.

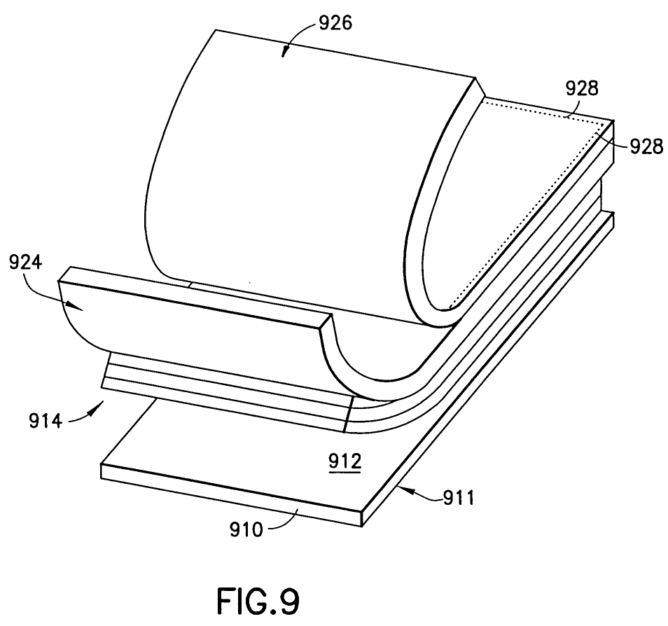

FIG. 9 illustrates the utilization of the SAP composite of the present invention in an absorbent pad. The absorbent pad comprises an outer layer 910 of a liquid impervious material, such as a thin plastic film or hydrophobic membrane, having an outer surface 911 and an inner surface 912. Positioned on the inner surface 912 of the outer layer 910 is at least one segment of the SAP composite of the present invention 914. In contact with the SAP composite 914 is a liquid acquisition layer 924. The acquisition layer 924 may be of any material currently used for this purpose and known to those skilled in the art. It may be, for example, an air laid medium including fibers forming a copious number of relatively large capillaries, or other absorptive materials. Completing the structure of FIG. 9 is an optional liquid-permeable skin-contacting spun-bonded medium 926.

The layers comprising the structure of FIG. 9 are bonded to one another by means such as a weld 928. The weld 928 extends about the periphery of and at least through the liquid acquisition layer 924, and the outer layer 910, attaching the acquisition layer 924 to the outer layer 910 and sandwiching the SAP composite 914 therebetween.

The invention is illustrated, but not limited, by the following examples.

### EXAMPLE 1

### Production of SAP Composite Sample

The composite sample was produced utilizing apparatus and methodology illustrated in FIG. 6. A mixture of 85.0% by weight SAP grade SP-1224 (Stockhausen Corporation, Greenboro, N.C., USA) was blended with 15.0% by weight FN510 FFS low-density polyethylene binder (Equistar Corporation, Houston, Tex.). An idler roller was heated to 425° F. and an infra-red pre-heater oven was heated to an emission temperature of approximately 700° F. A primary supply roll was loaded with a first substrate material in the form of cellulose tissue stock. A secondary supply roll was loaded with second substrate material in the form of cellulose tissue stock. The nip force was set at approximately 40-70 pounds per linear inch, and the linear web speed set to 5.0 feet/minute. The above SAP/binder mixture was applied to the first substrate in an amount of approximately 0.25 grams/square inch.

### COMPARATIVE EXAMPLE 1

### Production of Prior Art SAP with Fluff Pulp Composite Sample

In producing a prior art absorbent composite, a mixture of 74.0% by weight conventional SAP (BASF Corporation/Chemdahl grade L401 granular SAP, Mount Olive, N.J., USA) was blended with 16.0% by weight FN510 FFS low-density polyethylene binder, and 10.0% Solka Floc (FSD Corporation, Urbana, Ohio) fluff pulp. An idler roller was heated to 425° F. and an infra-red pre-heater oven was heated to an emission temperature of approximately 830° F. A primary supply roll was loaded with a first substrate material in the form of cellulose tissue stock. A secondary supply roll was loaded with second substrate material in the form of cellulose tissue stock. The nip force was set at approximately 40-80 pounds per linear inch, and the linear web speed set to 10.0 feet/minute. The above SAP/binder mixture was applied to the first substrate in an amount of approximately 0.30 grams/square inch.

### COMPARATIVE EXAMPLE 2

### Production of Prior Art SAP Only Composite Sample

In producing a prior art absorbent composite, a mixture of 74.0% by weight conventional SAP (BASF Corporation/Chemdahl grade L401 granular SAP, Mount Olive, N.J., USA) was blended with 16.0% by weight FN510 FFS low-density polyethylene binder. An idler roller was heated to 335° F. and an infra-red pre-heater oven was heated to an emission temperature of approximately 700° F. A primary supply roll was loaded with a first substrate material in the form of cellulose tissue stock. A secondary supply roll was loaded with second substrate material in the form of cellulose tissue stock. The nip force was set at approximately 70-80 pounds per linear inch, and the linear web speed set to 5.0 feet/minute. The above SAP/binder mixture was applied to the first substrate in an amount of approximately 0.30 grams/square inch.

### EXAMPLE 2

### Physical Structure of SAP Composites Following Wetting

Two 4.75″×4.75″ samples of each of the absorbent composite produced in Example 1 and Comparative Example above were exposed to a total liquid challenge of 100 ml of 1% saline in deionized water. For identification purposes, the liquid was amended with a small amount of common red food coloring. Following absorption of the challenge liquid, the second substrate (top sheet) of both samples were gently lifted and removed to reveal the structure of the resultant SAP composite following exposure of the composite to liquid.

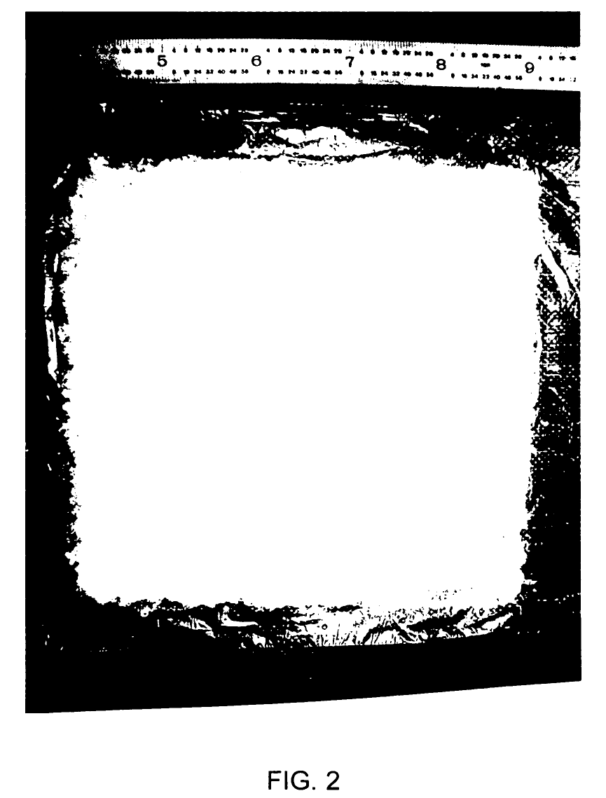

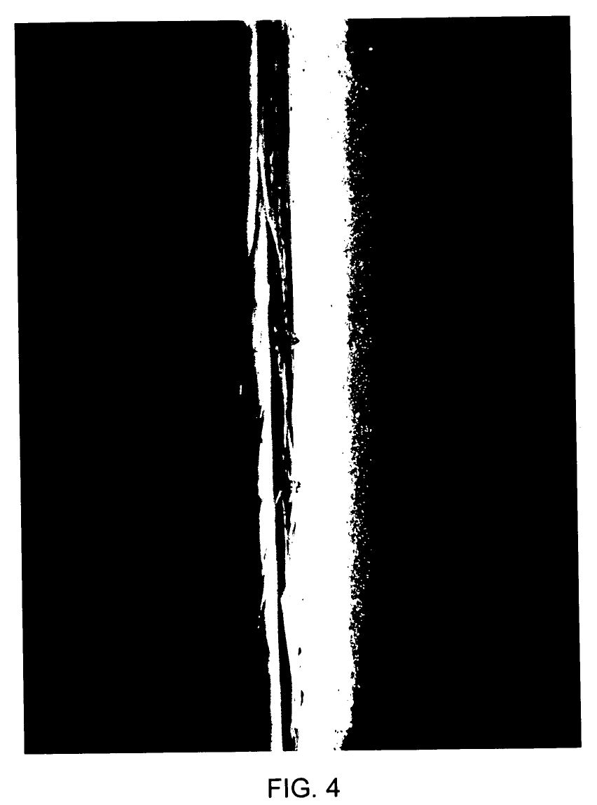

FIG. 1 and FIG. 3 show the three-dimensional array of channels that spontaneously form when the SAP composite of the present invention produced in Example 1 is brought into contact with liquid. FIG. 2 and FIG. 4 show the effect liquid contact has on a prior art SAP composite produced in the Comparative Example 2.

The channels shown in FIG. 1 and FIG. 3 spontaneously form within about the first 2-4 seconds after the SAP composite of the present invention is brought into contact with liquid. Rapid containment of liquid within the channels is immediately observed, followed by absorption of the liquid by the SAP particles within about 15-25 seconds following channel formation.

The formation of the channels as illustrated in FIG. 1 and FIG. 3 is markedly different from that which results from liquid contact of the prior art SAP composite of the Comparative Example, as is shown in FIG. 2 and FIG. 4. As FIG. 2 and FIG. 4 illustrate, when a prior art SAP composite is brought into contact with a liquid, the prior art SAP particles swell into a planar, expanded bed of swollen SAP particles. This process is initially slow, with a delay period of approximately 3-8 seconds wherein no obvious visual change in the sample is observed. This initial swelling is followed by a gradual absorption of the liquid over a period of approximately 30 seconds. When the prior art SAP composite of the Comparative Example was brought into contact with liquid, no channel formation, or enclosing of bulk liquid flow within channels was observed.

### EXAMPLE 3

### Absorption Testing of SAP Composite Sample from Example 1, and a Commercially Available Adult Incontinence Product

A 4.75″×4.75″ sample of the present invention SAP composite of Example 1, which had an average thickness of 0.975 mm and a mass of 5.6645 g, was tested in direct comparison with a commercially available incontinence pad, Depends® for Men, utilizing the apparatus and methodology illustrated in FIG. 7.

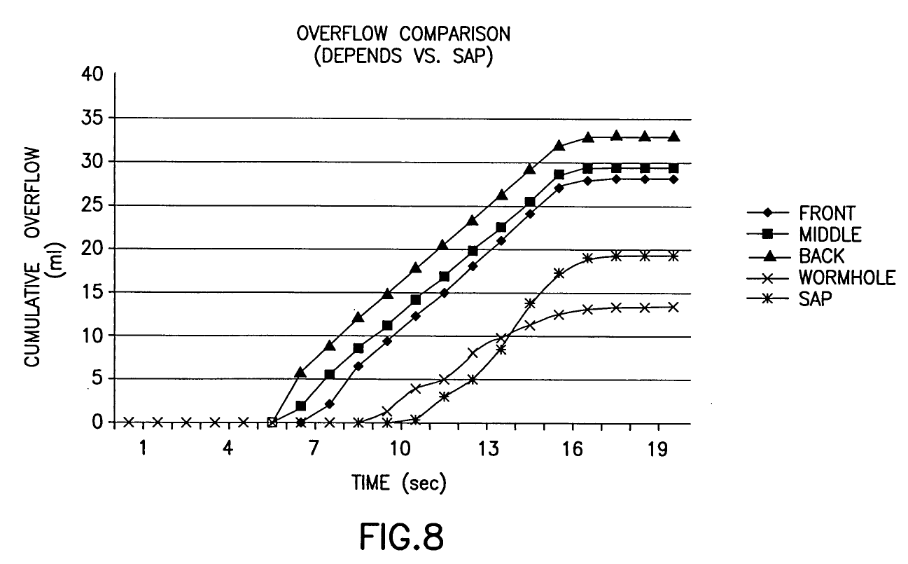

A liquid impermeable layer of plastic was placed over a hole in the plate to prevent the possibility of leakage. The composite of the present invention of Example 1 above was then accurately aligned over the plastic film, such that the square sample was equidistant from all sides of the hole. Two layers of a 4.750″×4.750″ polyester carded, nonwoven, high loft acquisition material were placed directly over the SAP composite of Example 1. A standard drop zone plate with a 1.0″ diameter chamber was then placed directly on top of the apparatus and a delivery tube was arranged to allow liquid to impinge the sample held under the plate within the drop zone. A total of 100 mls of liquid (1% aqueous saline) was delivered at 7 mls/second onto the SAP composite of Example 1. The overflow results are shown in FIG. 8.

The Depends® for Men incontinence pad was cut into three 4.750″×4.750″ sections representing the front, middle and back portions of the pad, as the pad is designed to fit on the human body. The samples had an average thickness of 4.650 mm and an average mass of 9.1853 g. The sample was accurately aligned as described above and the same test procedure and arrangement were effected. To assess possible variations between locations on the Depends® product, a total of three tests were conducted, one for each of the three separate locations on the Depends® for Men pad.

As indicated in FIG. 8, the overflow of the present invention, when combined with an acquisition layer similar to the one present on the Depends® for Men pad, outperforms the Depends® for Men sample, on average, by over 50%, and the Comparative Example 1 having prior art SAP by approximately 25%. This large improvement in absorbency of the present invention over the Depends® for Men pad is accomplished while reducing the thickness of the absorbent structure by 75%, along with a 50% reduction in sample mass.

The present invention describes liquid absorbent composites, more specifically described are disposable liquid absorbent composites with uses including, but not limited to, adult incontinence products, diapers, training pants, and paper towels.

Although the present invention has been described with respect to one or more particular embodiments, it will be understood that other embodiments of the present invention may be employed without departing from the spirit and scope of the present invention. Hence, the present invention is deemed limited only by the appended claims and the reasonable interpretation thereof.

1. A composite comprising:
a first substrate;
a bonded mixture, said bonded mixture comprising a mixture of binder particles and super-absorbent polymer particles, wherein said super-absorbent polymer particles having a property of forming a three-dimensional array of elongated channels upon contact with a liquid in the manner of SP-1224, and said binder particles are on average smaller than said super-absorbent polymer particles, and wherein at least some of said binder particles coalesce at least some of said super-absorbent polymer particles to each other and to said substrate; and
said three-dimensional array of elongated channels within said composite, formed by said super-absorbent polymer particles when in contact with a liquid, which promote liquid acquisition into said composite along the three-dimensional array of elongated channels prior to liquid absorption by the super-absorbent polymer particles.

2. The composite of claim 1, wherein at least some of said bonded mixture has the property of collecting liquid within said three-dimensional array, and the collected liquid in said array is absorbed by at least some of said bonded mixture.

3. The composite of claim 1, further comprising a second substrate, and said bonded mixture is between said first substrate and said second substrate, and wherein at least some of said binder particles coalesce at least some of said bonded mixture to said second substrate.

4. The composite of claim 1, wherein said bonded mixture has a dry thickness of less than about 2 millimeters.

5. The composite of claim 3, wherein said bonded mixture has a dry thickness of less than about 2 millimeters.

6. The composite of claim 1, wherein a liquid permeable acquisition layer is in liquid communication with said bonded mixture.

7. The composite of claim 3, wherein a liquid permeable acquisition layer is in liquid communication with said bonded mixture.

8. The composite of claim 1, wherein said first substrate is semi-permeable or impermeable to liquid.

9. The composite of claim 3, wherein said first substrate and said second substrate are either semi-permeable to liquid, impermeable to liquid, or a combination thereof.

10-16. (canceled)

17. A liquid absorbent pad which comprises:
an outer layer of a substantially liquid-impervious material having an outer surface and an inner surface;
at least one composite segment positioned on said inner surface of said liquid impervious material, said at least one composite segment comprising:
a first substrate and a bonded mixture, said bonded mixture comprising a mixture of binder particles and super-absorbent polymer particles, said super-absorbent polymer particles having a property of forming a three-dimensional array of elongated channels upon contact with a liquid in the manner of SP-1224, wherein said binder particles are on average smaller than said super-absorbent polymer particles, and wherein at least some of said binder particles coalesce at least some of said bonded mixture to said substrate; and
said three-dimensional array of elongated channels within said at least one composite segment formed after said at least one composite segment is contacted with a liquid; and
a liquid-permeable acquisition layer in liquid communication with said at least one composite segment, wherein at least a portion of said outer layer and said liquid permeable acquisition layer are directly or indirectly attached, and said at least one composite segment is sandwiched therebetween.

18. The liquid absorbent pad of claim 17, wherein said at least one composite segment further comprises a second substrate, and said bonded mixture is between said first substrate and said second substrate, and wherein at least some of said binder particles coalesce at least some of said bonded mixture to said second substrate.

19. The liquid absorbent pad of claim 17, wherein said at least one composite segment has a bonded mixture having a dry thickness of less than about 2 millimeters.

20. The liquid absorbent pad of claim 18, wherein said at least one composite segment has a bonded mixture having a dry thickness of less than about 2 millimeters.

21. A liquid absorbent pad comprising:
a substantially liquid-impervious material having an outer surface and an inner surface;
a composite positioned on the inner surface of said substantially liquid impervious material, said composite comprising a first substrate and a bonded mixture, the bonded mixture comprising a mixture of binder particles and super-absorbent polymer particles, wherein said super-absorbent polymer having a property of forming a three-dimensional array of elongated channels upon contact with a liquid in the manner of SP-1224, and said binder particles are on average smaller than the super-absorbent polymer particles, and wherein at least some of the binder particles coalesce at least some of the bonded mixture to the first substrate;
said three-dimensional array of elongated channels within said composite when said liquid absorbent pad is contacted with a liquid.

22. A liquid absorbent pad of claim 21 wherein the three-dimensional array of elongated channels within the composite acquire any liquid in contact with said liquid absorbent pad into said composite prior to absorption of the liquid by the super-absorbent polymer particles.

23. The liquid absorbent pad of claim 21, wherein said composite further comprises a second substrate, and said bonded mixture is between said first substrate and said second substrate, and wherein at least some of said binder particles coalesce at least some of said bonded mixture to said second substrate.

24. The liquid absorbent pad of claim 22, wherein said composite has a bonded mixture having a dry thickness of less than about 2 millimeters.

25. The liquid absorbent pad of claim 21, wherein said composite has a bonded mixture having a dry thickness of less than about 2 millimeters.
